(19) United States
(12) Patent Application Publication
(10) Pub. No.: US 2009/0264495 A1
(43) Pub. Date: Oct. 22, 2009

(54) ORAL SUSTAINED-RELEASE PHARMACEUTICAL COMPOSITION OF INDAPAMIDE, PRODUCTION AND USE THEREOF

(75) Inventors: Chunyi YEH, Sikou Township (TW); Yi-Lung WANG, Luchu Hsiang (TW); Ya-Sheng YANG, Hsinying City (TW); Ya-Ching CHANG CHIEN, Kaohsiung (TW)

Correspondence Address: PATENTTM.US, P. O. BOX 82788, PORTLAND, OR, 97282-0788, US

(73) Assignee: STANDARD CHEM. & PHARM. CO., LTD., HSIN-YING CITY (TW)

(21) Appl. No.: 12/493,002

(22) Filed: Jun. 26, 2009

Related U.S. Application Data
(63) Continuation-in-part of application No. 11/060,882, filed on Feb. 17, 2005, now abandoned.

(51) Int. Cl. A61K 31/405

(52) U.S. Cl. 514/415

(57) ABSTRACT

An oral sustained-release pharmaceutical composition of indapamide and a process for producing the foregoing pharmaceutical composition are provided. The pharmaceutical composition comprises indapamide in an amount between 0.2% and 4% (w/w) of the composition, a hydrophilic polymer in an amount between 10 % and 30% (w/w) of the composition, a dry binding agent in an amount between 2% and 20% (w/w) of the composition, and an erosion modifier in an amount between 40% and 80% (w/w) of the composition. The sustained-release pharmaceutical composition of indapamide could be obtained at lower expenditure and produced in a safe and economical manner.

### RELATED APPLICATION

The present invention is a Continuation-in-Part (CIP) of U.S. patent application Ser. No. 11/060,882 filed on Feb. 17, 2005, all of which are incorporated by reference in the present application in their entirety.

### BACKGROUND OF THE INVENTION

1. Field of the Invention

The present invention relates to an oral sustained-release pharmaceutical composition of indapamide, a process for producing the oral pharmaceutical composition and a pharmaceutical composition produced by the process.

2. Description of Related Art

Indapamide is a sulfonamide derivative with an indole ring, which has been used as a diuretic. Indapamide inhibits the reabsorption of sodium in the cortical dilution segment and increases the urinary excretion of sodium and chloride, thereby increase urine output and has an antihypertensive action. However, administering a conventional oral immediate-release tablet of indapamide to a patient in need of therapy rapidly increases the concentration of indapamide in the blood of the patient, and a “peak effect” occurs, which causes some undesired side effects.

U.S. Pat. No. 5,334,392 disclosed a conventional sustained-release 22 formulation of indapamide, which comprises 1 to 2.5 mg of indapamide, polyvidone, methylhydroxyalkyl cellulose and excipients. The formulation 24 according to U.S. Pat. No. 5,334,392 was produced by admixing about 1% of indapamide with 2-10% of polyvidone of a molecular weight between 10,000 and 700,000, sugar and colloidal silica, followed by wetting with an aqueous alcoholic solution to form granules. After drying, the granules were admixed with 30-50% of methylhydroxyalkyl cellulose with a viscosity between 1,000 and 20,000 centipoises (cps). After addition of lubricants, tablets were obtained with a hardness of 60 to 75 N (6.12 to 7.65 kp) as measured by diametrical crushing. However, polyvidone is so moisture-absorbing the tablets so produced are less stable. Furthermore, flame-proof apparatuses are required during the manufacturing process because of use of ethanol, which must be added to carry out the subsequent granulation. Apart from a complicated and unsafe manufacturing process, the resultant tablets unavoidably contain organic solvents as impurities.

In an attempt to the resolve the foregoing problems, WO 2004/002475 A1 disclosed a manufacturing process where the organic solvents were replaced with water. The obtained sustained-release indapamide formulation includes 1.5%-2.5% indapamide, 30-80% lactose monohydrate, 2-10% copovidon, 20-65% hypromellose and 0.1-5% lubricants. The substitute use of water for ethanol made the manufacturing process much safer. However, when hypromellose dissolved with water, it became a high viscosity solution, renders the powder so sticky and made the granulation process getting more difficult. The drying process followed by the granulation process not only takes much time, but also need lots of energy.

To obtain a better therapeutic index at lower expenditure, there is still a need of a sustained-release pharmaceutical composition of indapamide in the art, which is produced in a safe and economical manner.

### SUMMARY OF THE INVENTION

One aspect of the present invention relates to an oral pharmaceutical composition for prolonged sustained-release of indapamide so the concentration of indapamide in the blood is more stable over a period. Patients only have to take one dose of the oral pharmaceutical composition in accordance with the present invention in a 24-hour period, which decreases undesired side effects and maintains a stable concentration of indapamide in the blood to obtain a better therapeutic index. Meanwhile, the sustained-release pharmaceutical composition of indapamide could be obtained at lower expenditure and produced in a safe and economical manner.

Preferably, the oral sustained-release pharmaceutical composition of indapamide in accordance with the present invention comprises indapamide in an amount between 0.2% and 4% (w/w) of the composition, a hydrophilic polymer in an amount between 10% and 30% (w/w) of the composition, a dry binding agent in an amount between 2% and 20% (w/w) of the composition, and an erosion modifier in an amount between 40% and 80% (w/w) of the composition.

Preferably, the “hydrophilic polymer” that may be employed in accordance with the present invention includes polyethylene oxide, hydroxypropyl methyl cellulose, polyvinyl alcohol or a combination thereof. The hydrophilic polymer employed in accordance with the present invention preferably has a viscosity between 25,000 and 300,000 centipoises (cps); more preferably, between 30,000 and 100,000 cps.

Preferably, the hydroxypropyl methyl cellulose contains 22.0˜24.0% methyl group and 8.0˜12.0% hydroxyl-propoxyl group.

Another aspect of the present invention relates to a process to produce an oral sustained-release pharmaceutical composition of indapamide, which comprises a simple mixing instead of complicated steps of conventional wet granulation.

Preferably, the process in accordance with the present invention comprises mixing together indapamide in an amount between 0.2% and 4% (w/w) of the composition with a hydrophilic polymer in an amount between 10% and 30% (w/w) of the composition, a dry binding agent in an amount between 2% and 20% (w/w) of the composition and an erosion modifier in an amount between 40% and 80% (w/w) of the composition to obtain a mixture, and compressing the mixture into tablets.

Preferably, the “dry binding agent” that may be employed in accordance with the present invention includes hydroxyethyl cellulose, hydroxyproxyl cellulose and pregelatinized starch.

Preferably, the “erosion modifier” that may be employed in accordance with the present invention contains a hydrophilic erosion accelerator, a hydrophobic erosion inhibitor or a combination thereof. The hydrophilic erosion accelerator is preferably, but not limited to, sugar, lactose, glucose, maltose or mannitol. The hydrophobic erosion inhibitor is preferably, but not limited to, silicate, phosphate, carbonate, glyceryl behenate or sterate.

The oral pharmaceutical composition in accordance with the present invention preferably provides a sustained release of the indapamide for about 24 hours after the composition is administered. The mixture is preferably compressed in a rotary tableting machine to obtain round-shape tablets of a diameter of about 8 mm and with hardness of about 4 to 12 kp; and preferably, 4 to 9 kp.

More preferably, the tablets are coated with a light-resistant film. Most preferably, the light-resistant film is selected from the group consisting of hydroxypropyl methyl cellulose, polyethylene glycol and titanium dioxide.

Preferably, the oral pharmaceutical composition in accordance with the present invention may be in the form of a tablet or a capsule.

By using hydrophilic polymer, such as hydroxypropyl methyl cellulose, acquiring lower equilibrium moisture content, the oral sustained-release pharmaceutical composition of indapamide in accordance with the present invention using direct compaction process has lower moisture content and higher stability than conventional preparation. Moreover, in absence of using vaporizable organic solvent, the oral sustained-release pharmaceutical composition of indapamide in accordance with the present invention contains no organic solvents as impurities.

Furthermore, the hypromellose used in present invention is suitable for direct compaction without granulation process, such that operation and control of the subsequent procedures for manufacturing the oral pharmaceutical composition for sustained-release of indapamide in accordance with the present invention are much easier.

Further benefits and advantages of the present invention will become apparent after a careful reading of the detailed description.

### BRIEF DESCRIPTION OF THE DRAWINGS

FIG. 1 is a diagram showing dissolution profiles of sustained-release indapamide formulations in accordance with the present invention, with a conventional indapamide formulation as the control group.

FIG. 2 is a diagram showing dissolution profiles of sustained-release indapamide formulations in accordance with the present invention, with a conventional indapamide formulation as the control group.

FIG. 3 is a diagram showing the profile of blood concentration of indapamide after administering a sustained-release indapamide formulation in accordance with the present invention to patients, with a conventional indapamide formulation as the control group.

### DETAILED DESCRIPTION OF HE PREFERRED EMBODIMENTS

The term “sustained release” used in accordance with the present invention refers to formulations or dosage units of indapamide that are slowly and continuously dissolved and absorbed in the stomach and gastrointestinal tract over a period of time.

Hydrophilic polymer will swell sufficiently in the gastrointestinal tract and then become gel-like. Hydrophilic polymer which may be employed in accordance with the present invention includes derivatives of cellulose, in particular cellulose ethers such as hydroxypropyl cellulose, hydroxymethyl cellulose, methyl cellulose or methyl hydroxypropyl cellulose, and among different commercial grades of these ethers, those showing relatively high viscosities are preferred.

Dry binding agent is used to assist the binding to the hydrophilic polymer and erosion modifier in the molding step. Dry binding agent which may be employed in accordance with the present invention include hydroxyethyl cellulose, hydroxyproxyl cellulose and pregelatinized starch.

Erosion modifier contains a hydrophilic erosion accelerator, a hydrophobic erosion inhibitor or a combination thereof. A ratio of the hydrophilic erosion accelerator and the hydrophobic erosion inhibitor controls rate of sustained release.

The oral sustained-release pharmaceutical composition of indapamide in accordance with the present invention provides prolonged sustained release of indapamide so the concentration of indapamide in the blood is more stable.

Therefore, patients in need of indapamide-related therapies only have to take one dose of the pharmaceutical composition in accordance with the present invention in a 24-hour period, which decreases undesired side effects and sustains a stable concentration of indapamide in the blood.

### EXAMPLES

### Example 1

### Sustained-Release Indapamide Formulation (1) and a Process for the Production Thereof

1.1 Material

Sustained-release indapamide formulation (1) was shown in the following table:

TABLE 1a
Sustained-release indapamide formulation (1)

| Composition | Amount (mg) |
|---|---|
| Indapamide | 1.5 |
| Lactose | 112.5 |
| Dicalcium phosphate | 30 |
| Hydroxypropyl methyl cellulose (100,000 cps) | 40 |
| Hydroxyproxyl cellulose | 10 |
| Silicon dioxide | 3 |
| Magnesium stearate | 3 |

Hydroxyporpyl methyl cellulose was Substitution Type 2208 (Metolose 90SH-100000SR, Shin Etsu) as defined in the following table.

TABLE 1b
The property of Hydroxyporpyl methyl cellulose used in the present
example

| | | | Substitution content | | Mean |
|---|---|---|---|---|---|
| | | Labeled | | Hydroxy- | particle |
| Description | Grade | viscosity (cps) | Methoxyl (%) | propoxyl (%) | size (μm) |
| Hydroxypropyl | 90SH- | 100,000 | 22.0~24.0% | 8.0~12.0% | 50 |
| methyl cellulose | 100000SR | | | | |

1.2 Process

An oral sustained-release pharmaceutical composition of indapamide was prepared by steps as following:

Step 1: Indapamide, lactose, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tablets having a diameter of approximately 8 mm and a hardness of approximately 4 to 9 kp.

### Example 2

### Sustained-Release Indapamide Formulation (2) and a Process for the Production Thereof

2.1 Material

Sustained-release indapamide formulation (2) was shown in the following table:

TABLE 2a
Sustained-release indapamide formulation (2)

| Composition | Amount (mg) |
|---|---|
| Indapamide | 1.5 |
| Lactose | 72.5 |
| Dicalcium phosphate | 80 |
| Hydroxypropyl methyl cellulose (100,000 cps) | 30 |
| Hydroxyproxyl cellulose | 10 |
| Silicon dioxide | 3 |
| Magnesium stearate | 3 |

Hydroxypropyl methyl cellulose was Substitution Type 2208 (Metolose 90SH-100000SR, Shin Etsu) as defined in the following table.

TABLE 2b
The property of Hydroxyporpyl methyl cellulose used in the present
example

| | | | Substitution content | | Mean |
|---|---|---|---|---|---|
| | | Labeled | | Hydroxy- | particle |
| Description | Grade | viscosity (cps) | Methoxyl (%) | propoxyl (%) | size (μm) |
| Hydroxypropyl | 90SH- | 100,000 | 22.0~24.0% | 8.0~12.0% | 50 |
| methyl cellulose | 100000SR | | | | |

2.2 Process

An oral sustained-release pharmaceutical composition of indapamide was prepared by steps as following:

Step 1: Indapamide, lactose, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tablets having a diameter of approximately 8 mm and a hardness of approximately 4 to 9 kp.

### Example 3

### Sustained-Release Indapamide Formulation (3) and Method for the Production Thereof

3.1 Material

Sustained-release indapamide formulation (3) was shown in the following table:

TABLE 3a
Sustained-release indapamide formulation (3)

| Composition | Amount (mg) |
|---|---|
| Indapamide | 1.5 |
| Lactose | 115.0 |
| Hydroxypropyl methyl cellulose (100,000 cps) | 47.5 |
| Pregelatinized starch | 30.0 |
| Silicon dioxide | 3.0 |
| Magnesium stearate | 3.0 |

Hydroxypropyl methyl cellulose was Substitution Type 2208 (Metolose 90SH-100000SR, Shin Etsu) as defined in the following table.

TABLE 3b
The property of Hydroxyporpyl methyl cellulose used in the present
example

| | | | Substitution content | | Mean |
|---|---|---|---|---|---|
| | | Labeled | | Hydroxy- | particle |
| Description | Grade | viscosity (cps) | Methoxyl (%) | propoxyl (%) | size (μm) |
| Hydroxypropyl | 90SH- | 100,000 | 22.0~24.0% | 8.0~12.0% | 50 |
| methyl cellulose | 100000SR | | | | |

3.2 Process

An oral sustained-release pharmaceutical composition of indapamide was prepared by steps as following:

Step 1: Indapamide, lactose, hydroxypropyl methyl cellulose, starch, silicon dioxide and magnesium sterate were mixed well.

Step 2: The mixture in step 1 was compressed in a rotary tableting machine to obtain tablets having a diameter of approximately 8 mm and a hardness of approximately 4 to 9 kp.

### Example 4

### Sustained-Release Indapamide Formulation (4) and Method for the Production Thereof

4.1 Material

Sustained-release indapamide formulation (4) was shown in the following table:

TABLE 4a
Sustained-release indapamide formulation (4)

| Composition | Amount (mg) |
|---|---|
| Indapamide | 1.5 |
| Mannitol | 58.5 |
| Dicalcium phosphate | 60 |
| Hydroxypropyl methyl cellulose (30,000 cps) | 58 |
| Hydroxyproxyl cellulose | 16 |
| Silicon dioxide | 3 |
| Magnesium stearate | 3 |

Hydroxypropyl methyl cellulose was (Metolose 90SH-30000SR, Shin Etsu) as defined in the following table.

TABLE 4b
The property of Hydroxyporpyl methyl
cellulose used in the present example

| | | Labeled | Substitution content | |
|---|---|---|---|---|
| | | viscosity | Methoxyl | Hydroxy- |
| Description | Grade | (cps) | (%) | propoxyl (%) |
| Hydroxypropyl | 90SH- | 30,000 | 1.4 | 0.20 |
| methyl cellulose | 30000 | | | |

4.2 Process

An oral sustained-release pharmaceutical composition of indapamide was prepared by steps as following:

Step 1: Indapamide, mannitol, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tablets having a diameter of approximately 8 mm and a hardness of approximately 4 to 9 kp.

### Example 5

### Sustained-Release Indapamide Formulation (5) and Method for the Production Thereof

5.1 Material

Sustained-release indapamide formulation (5) was shown in the following table:

TABLE 5a
Sustained-release indapamide formulation (5)

| Composition | Amount (mg) |
|---|---|
| Indapamide | 1.5 |
| Lactose | 132.5 |
| Dicalcium phosphate | 2.5 |
| Hydroxypropyl methyl cellulose (100,000 cps) | 47.5 |
| Hydroxyproxyl cellulose | 10 |
| Silicon dioxide | 3 |
| Magnesium stearate | 3 |

Hydroxypropyl methyl cellulose was Substitution Type 2208 (Metolose 90SH-10000SR, Shin Etsu) as defined in the following table.

TABLE 5b
The property of Hydroxyporpyl methyl cellulose used in the present
example

| | | | Substitution content | | Mean |
|---|---|---|---|---|---|
| | | Labeled | | Hydroxy- | particle |
| Description | Grade | viscosity (cps) | Methoxyl (%) | propoxyl (%) | size (μm) |
| Hydroxypropyl | 90SH- | 100,000 | 22.0~24.0% | 8.0~12.0% | 50 |
| methyl cellulose | 100000SR | | | | |

5.2 Process

An oral sustained-release pharmaceutical composition of indapamide was prepared by steps as following:

Step 1: Indapamide, lactose, dicalcium phosphate, hydroxypropyl methyl cellulose, hydroxyproxyl cellulose, silicon dioxide and magnesium sterate were mixed well.

Step 2: The mixture in step 1 was compressed in a rotary tableting machine to obtain tablets having a diameter of approximately 8 mm and a hardness of approximately 4 to 9 kp.

Step 3: Tablets in step 2 were coated film with 4.0 mg hydroxypropyl methyl cellulose, 0.75 mg polyethylene glycol and 0.25 mg titanium dioxide.

### Example 6

### Ingredient-Releasing Rate Test

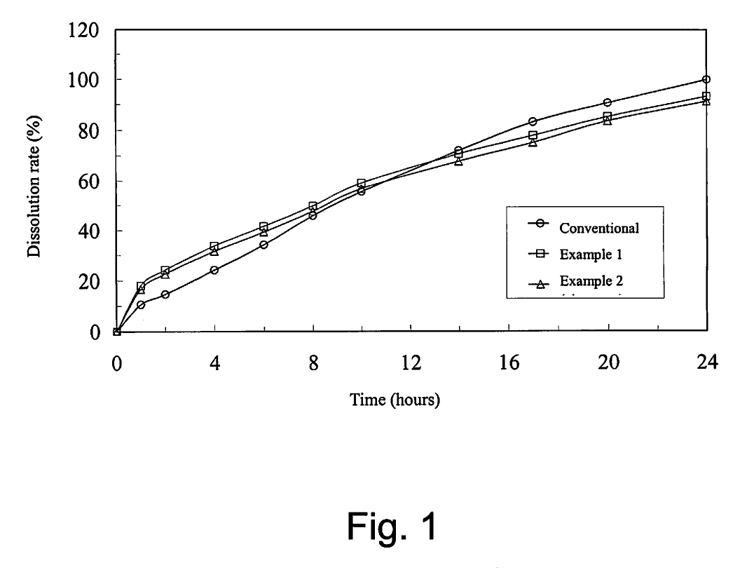

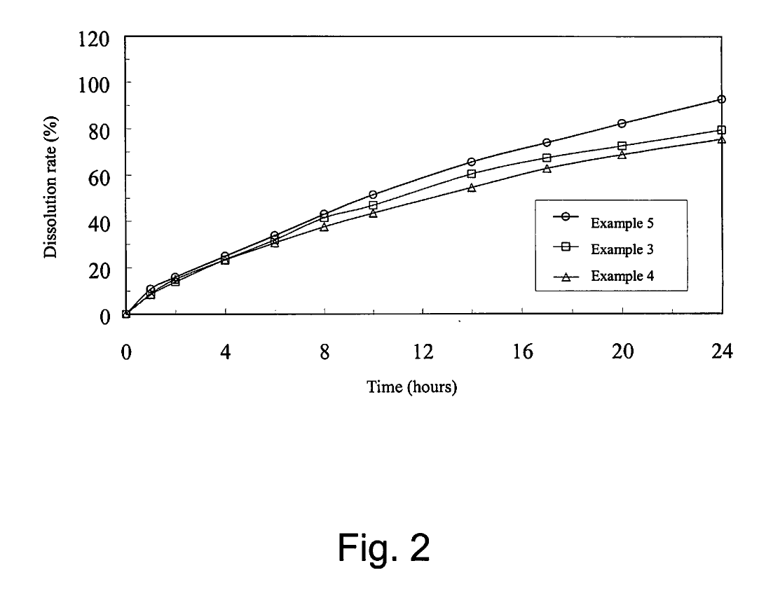

Rates of indapamide release from the indapamide formulations made according to Examples 1-5 and a conventional indapamide having an equivalent quantity of indapamide were evaluated in a dissolution test in accordance with instructions in the United States Pharmacopoeia (U.S.P) 27^{th} edition. In this test, each indapamide formulation and 500 mL of pH 6.8 phosphate buffer were poured into a vessel and heated to 37±0.5° C. Then, the mixture was stirred in a mixer at 75 rpm. The results are provided in Table 6 and FIGS. 1 and 2.

TABLE 6
Dissolution rate (%)

| Time | Conven- | Exam- | Exam- | Exam- | | |
|---|---|---|---|---|---|---|
| (hours) | tional | ple 1 | ple 2 | ple 3 | Example 4 | Example 5 |
| 0 | 0 | 0 | 0 | 0 | 0 | 0 |
| 1 | 10.68 | 18.06 | 16.62 | 8.43 | 8.73 | 10.90 |
| 2 | 14.67 | 24.24 | 22.71 | 13.95 | 15.14 | 16.00 |
| 4 | 24.22 | 33.82 | 31.68 | 23.46 | 23.38 | 24.92 |
| 6 | 34.28 | 41.70 | 39.43 | 31.95 | 30.65 | 33.79 |
| 8 | 45.92 | 49.90 | 47.72 | 41.55 | 37.63 | 43.05 |
| 10 | 55.54 | 59.05 | 56.80 | 46.93 | 43.55 | 51.48 |
| 14 | 72.05 | 70.75 | 67.81 | 60.44 | 54.63 | 65.60 |
| 17 | 83.28 | 78.03 | 75.25 | 67.39 | 62.82 | 73.97 |
| 20 | 90.78 | 85.33 | 83.69 | 72.55 | 68.77 | 82.19 |
| 24 | 100.0 | 93.33 | 91.36 | 79.50 | 75.50 | 92.69 |

The results in Table 1 and the data in FIGS. 1 and 2 show that all the indapamide formulations have stable sustained release efficacy in the pH 6.8 phosphate buffer. The ratios of the indapamide released from the formulations achieved 75% after 24 hours.

### Example 7

### The Blood Concentration of Sustained-Release Indapamide

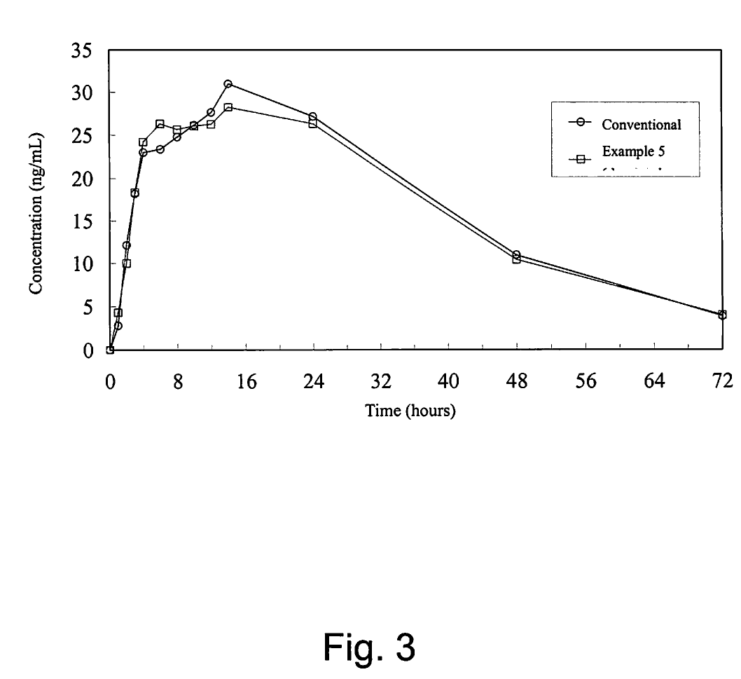

The blood concentration of indapamide was measured in five patients after taking tablets containing 1.5 mg indapamide in example 5 and conventional indapamide tablets. The data shown in Table 7 and FIG. 3 demonstrate that release of the indapamide formulations in accordance with the present invention can be controlled for more than 24 hours.

TABLE 7
Blood concentrations of indapamide (ng/mL)

| Time(Hours) | Conventional | Example 5 |
|---|---|---|
| 0 | 0 | 0 |
| 1.0 | 2.79 | 4.30 |
| 2.0 | 12.12 | 10.01 |
| 3.0 | 18.20 | 18.38 |
| 4.0 | 23.00 | 24.22 |
| 6.0 | 23.38 | 26.30 |
| 8.0 | 24.78 | 25.66 |
| 10.0 | 26.16 | 26.06 |
| 12.0 | 27.64 | 26.26 |
| 14.0 | 30.98 | 28.24 |
| 24.0 | 27.16 | 26.32 |
| 48.0 | 10.98 | 10.44 |
| 72.0 | 3.87 | 4.02 |

Accordingly, advantages of the sustained-release indapamide formulations in accordance with the present invention include the following.
1. Patients can reduce frequency of ingesting indapamide formulations.
2. The blood concentration of indapamide fluctuates less so that antinociceptive tolerance of patients is promoted.

Although the invention has been explained in relation to its preferred embodiment, many other possible modifications and variations can be made without departing from the spirit and scope of the invention as hereinafter claimed.

1. An oral sustained-release pharmaceutical composition of indapamide comprising:
indapamide in an amount between 0.2% and 4% (w/w) of the composition,
a hydrophilic polymer in an amount between 10 and 30% (w/w) of the composition, wherein viscosity of the hydrophilic polymer is between 2,5000 centipoises (cps) and 200,000 cps,
a dry binding agent in an amount between 2 and 20 (w/w) of the composition, and
an erosion modifier in an amount between 40 and 80 (w/w) of the composition.

2. The oral pharmaceutical composition as claimed in claim 1, wherein the hydrophilic polymer is selected from the group consisting of polyethylene oxide, hydroxypropyl methyl cellulose and polyvinyl alcohol.

3. The oral pharmaceutical composition as claimed in claim 1, wherein the hydrophilic polymer has a viscosity between 30,000 cps and 100,000 cps.

4. The oral pharmaceutical composition as claimed in claim 1, wherein hydroxypropyl methyl cellulose contains 22.0˜24.0% of methyl group and 8.0˜12.0% hydroxyl-propoxyl group.

5. The oral pharmaceutical composition as claimed in claim 1, wherein the dry binding agent is selected from the group consisting of hydroxyethyl cellulose, hydroxyproxyl cellulose and pregelatinized starch.

6. The oral pharmaceutical composition as claimed in claim 1, wherein the erosion modifier contains a hydrophilic erosion accelerator, hydrophobic erosion inhibitor or a combination thereof.

7. The oral pharmaceutical composition as claimed in claim 6, wherein the hydrophilic erosion accelerator is selected from the group consisting of sugar, lactose, glucose, maltose and mannitol.

8. The oral pharmaceutical composition as claimed in claim 6, wherein the hydrophobic erosion inhibitor is selected from the group consisting of silicate, phosphate, carbonate, glyceryl behenate and sterate.

9. The oral pharmaceutical composition as claimed in claim 1, which is in a form of a tablet or a capsule.

10. The oral pharmaceutical composition as claimed in claim 1, which is in a form of a round-shape tablet that has a diameter of about 8 mm and a hardness of about 4 to 9 kp.

11-23. (canceled)
